(19) United States
(12) Patent Application Publication
(10) Pub. No.: US 2006/0142180 A1
(43) Pub. Date: Jun. 29, 2006

(54) Cholinergic modulation of microglial activation via alpha-7 nicotinic receptors

(76) Inventors: Doug Shytle, Lutz, FL (US); Jun Tah, Tampa, FL (US); Paul R. Sanberg, Spring Hill, FL (US)

Correspondence Address: SMITH HOPEN, PA, 180 PINE AVENUE NORTH, OLDSMAR, FL, 34677, US

(21) Appl. No.: 11/349,455

(22) Filed: Feb. 7, 2006

Related U.S. Application Data
(63) Continuation of application No. PCT/US04/25714, filed on Aug. 9, 2004.
(60) Provisional application No. 60/481,192, filed on Aug. 7, 2003.

(51) Int. Cl. A61K 38/54; A61K 31/13

(52) U.S. Cl. 514/2; 514/663

(57) ABSTRACT

Novel therapeutic targets in the treatment of neuroimmunological and neurodegenerative disorders. Accordingly, methods of treating a neurodegenerative disorder in a patient, as well as inhibiting the release of a proinflammatory cytokine, comprising the step of contacting a target cell with a therapeutically effective amount of a cholinergic agonist, such as those chosen from the group consisting of acetycholine, nicotine, choline, galantamine, cytisine, GTS-21, or derivatives thereof, wherein the target cell is a microglia is provided.

### CROSS REFERENCE TO RELATED APPLICATIONS

This application is a continuation of PCT Application No. PCT/US2004/025714, filed Aug. 9, 2004, which claims benefit of U.S. Provisional Application No. 60/481,192, filed Aug. 7, 2003.

### BACKGROUND OF THE INVENTION

While considerable empirical evidence suggests that nicotine is neuroprotective in animal models of neurodegenerative disorders, the mechanism(s) of action are unclear. One area that has received little attention is the role of nicotinic acetylcholine receptors (nAChR) function in neuroimmunology. Almost all degenerative diseases of the central nervous system are connected to chronic inflammation and a central step in this process is the activation of brain mononuclear phagocyte cells, called microglia. Microglia represent a major cellular component of the brain, where they constitute a widely distributed network of immunoprotective cells.

Because acetylcholinergic neurons are particularly susceptible to the neurodegenerative consequences of microglial activation, the effects of ACh and nicotine on LPS-induced TNF-α release in mouse cultured microglial cells were used for the development of the present invention.

The inventors investigated the role of acetylcholine (ACh) in microglial activation induced by bacterial endotoxin, lypopolysaccharide (LPS). ACh and nicotine pretreatment inhibited LPS-induced TNF-α release in murine derived microglial cells, an effect prevented by nonselective nicotinic antagonist, mecamylamine, and by α7 selective nicotinic antagonist, α-bungurotoxin. Our findings uncover a cholinergic pathway that regulates microglial activation through α7 nicotinic receptor subtype. Preliminary findings indicate that downstream processes of this cholinergic effect do not involve kinase pathways traditionally thought to be critical for LPS-induced microglial activation. Therefore, this invention provides for novel therapeutic targets in the treatment of neuroimmunological and neurodegenerative disorders. Accordingly, the invention discloses that the microglial α7 nicotinic receptor is an important novel target for treating disorders involving neuroinflammation, such as Alzheimer's, Parkinson's, ALS, Downs Syndrome, etc.

### SUMMARY OF INVENTION

The present invention includes a method of treating a neurodegenerative disorder in a patient, comprising the step of contacting a target cell with a therapeutically effective amount of a cholinergic agonist, such as those chosen from the group consisting of acetycholine and nicotine, wherein the target cell is a microglia.

Another embodiment of the invention includes a method if inhibiting the release of Tumor Necrosis Factor (alpha) from a cell, comprising the step of contacting the cell with a therapeutically effective amount of a cholinergic agonist selected from the group consisting of acetycholine and nicotine. Additional nicotine receptor agonists of interest include, but are not necessarily limited to, naturally occurring plant alkaloids (e.g., galantamine, galantamine derivatives, cytisine, cytisine derivatives, and the like), which plant derived compounds can be provided in a herbal preparation (e.g., in the form of dried tobacco leaves, in a poultice, in a botanical preparation, etc.), in isolated form (e.g., separated or partially separated from the materials that naturally accompany it), or in a substantially purified form. Other nicotine receptor agonists include cholineesterase inhibitors (e.g., that increase local concentration of acetylcholine), derivatives of nicotine analogues that specifically bind the neuronal type of nicotinic receptors (with reduced binding to the muscarinic receptor) and having reduced deleterious side-effects (e.g., Epidoxidine, ABT-154, ABT-418, ABT-594, ABT-089; Abbott Laboratories (Damaj et al. (1998) J. Pharmacol Exp. Ther. 284:1058-65; describing several analogs of epibatidine of equal potency but with high specificity to the neuronal type of nicotinic receptors). Further nicotine receptor agonists of interest include, but are not necessarily limited to, N-methylcarbamyl and N-methylthi-O-carbamyl esters of choline (e.g., trimethylaminoethanol) (Abood et al. (1988) Pharmacol. Biochem. Behav. 30:403-8); GTS-21 (Stokes et al., Mol Pharmacol. 2004, 66(1):14-24) and the like.

### BRIEF DESCRIPTION OF THE DRAWINGS

For a fuller understanding of the nature and objects of the invention, reference should be made to the following detailed description, taken in connection with the accompanying drawings, in which:

FIG. 1. shows immunoblots and graphs demonstrating that the nAChRα7 subunit is expressed by microglia.

FIG. 2. shows that α-bungarotoxin, a α7 nAChR subunit-selective blocker, can bind to this receptor and be displaced by nicotine.

FIG. 3. shows graphs indicating that the microglial nAChR α7 subunit is functional. Graphs represent the summary of TNF-α release ELISA results (mean TNF-α pg/mg of total protein±1 SEM) with n=3 for each condition presented.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

As used herein, a cytokine is a soluble protein or peptide which is naturally produced by mammalian cells and which, either under normal or pathological conditions, modulate the functional activities of individual cells and tissues. A proinflammatory cytokine is a cytokine that is capable of causing any of the following physiological reactions associated with inflammation: vasodialation, hyperemia, increased permeability of vessels with associated edema, accumulation of granulocytes and mononuclear phagocytes, or deposition of fibrin. In one embodiment of the invention, the proinflammatory cytokine that is inhibited by cholinergic agonist treatment is TNF-α. The present invention addresses the role of acetylcholine (ACh) in microglial activation induced by bacterial endotoxin, lypopolysaccharide (LPS). The pretreatment with either ACh or nicotine markedly inhibited LPS-induced TNF-α release in murine derived microglial cells. This effect was attenuated by co-pretreatment with the α7 selective nicotinic antagonist, α-bungarotoxin. Accordingly, the present invention discloses a cholinergic pathway that regulates microglial activation through α7 nicotinic receptor subtype.

Over the last five years there has been a rapid accumulation of evidence suggesting that neuronal nicotinic acetylcholine receptors (nAChRs) play important roles in neurodegenerative diseases. First, there is a well-established loss of nAChRs in post-mortem brains from patients with Alzheimer's disease (AD), Parkinson's disease (PD) and a range of other neurodegenerative disorders. This loss is generally more substantial than the reduction found in muscarinic cholinergic receptors and contrasts, perhaps in a functionally relevant way, with the higher density of nAChRs reported present in the brain of normal smokers. Second, epidemiologic studies have reported a consistent inverse relationship between nicotine intake (from tobacco smoking) and the incidence of both PD and AD, suggesting that smoking may be neuroprotective. Fourth, the degree of cognitive impairment found in AD correlates well with the central cholinergic deficiency and treatment with nAChR agonists result in long-lasting improvement of cognitive performance in aging rats, monkeys, and humans.

Considerable empirical evidence suggests that nicotine is neuroprotective in animal models of neurodegenerative disorders. Both in vitro and in vivo, nicotine protects striatal, hippocampal, and cortical neurons against the neurotoxicity induced by excitotoxic amino acids as well as the toxicity caused by β-amyloid, the major component of senile plaques of AD. The neuroprotective effects of nicotine appear to be mediated mainly by α7 and α4β2 nAChR subtypes, which are involved in a variety of neuronal cellular functions ranging from the presynaptic release of various neurotransmitters and growth factors to a reduction of superoxide anion generation.

### EXAMPLE 1

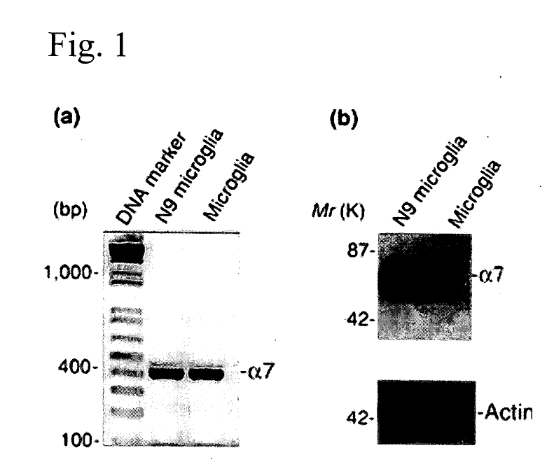

Murine primary culture microglial cells were isolated from mouse cerebral cortices (C57BL/6 mice) and were cultured using the methods set forth by Tan, J. et al. (J Neurosci 20, 7587-94 (2000). Murine primary culture microglial cells were isolated from mouse cerebral cortices (C57BL/6 mice) and cultured as previously described (Tan et al. 2000b). To investigate whether α7 nAChR subunits are expressed in microglial cells, we first isolated total RNA from N9 microglial cell line and primary cultured microglial cells for RT-PCR analysis. Results show that the α7 nAChR subunit mRNA was detected in both of these cells (FIG. 1a). Further, α7 nAChR subunit protein was detected in primary cultured microglial cells by western blot (FIG. 1b).

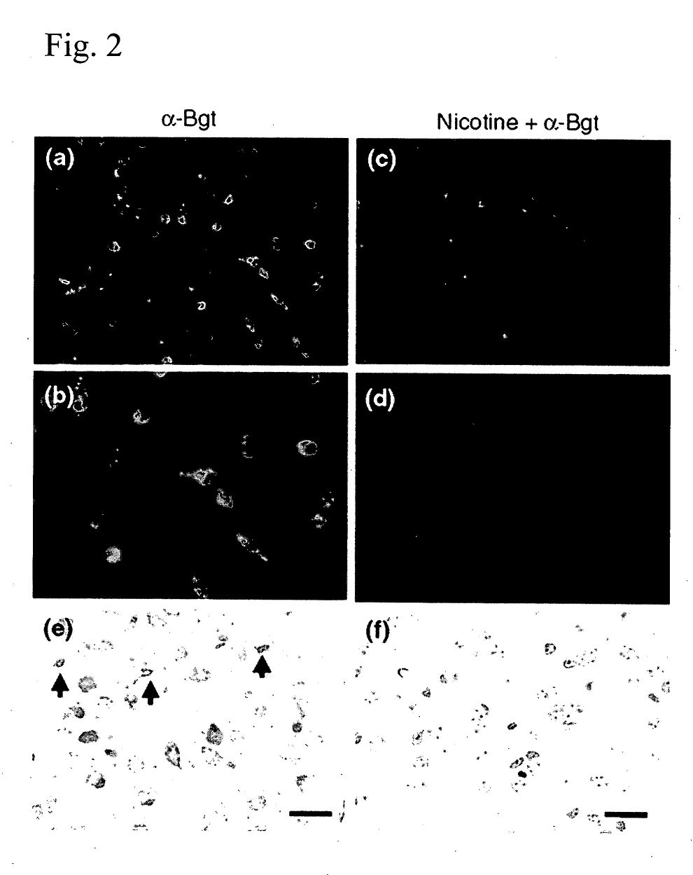

To evaluate whether α-bungarotoxin, a α7 nAChR subunit-selective blocker, could bind to this receptor, we pre-treated primary microglial cells in the presence or absence of nicotine and then incubated with FITC-labeled α-bungarotoxin. As shown in FIGS. 2(c and d), nicotine pre-treatment resulted in a marked reduction of fluorescent intensity of FITC-labeled α-bungarotoxin binding compared to the absence of nicotine (FIGS. 2a and b). In order to gain further insight into the expression of α7 nAChR in situ, we performed immunohistochemistry on adult mouse brain and found that microglial cells stain positively for α7 nAChR subunit (FIG. 2e). To rule out the possibility of non-specific binding, PBS was used instead of primary antibody as a negative control (FIG. 2f). Additionally, we also stained cells using normal rabbit serum (the appropriate isotype control) instead of rabbit anti α7 AChR primary antibody and results were similar to the PBS-negative control (data not shown).

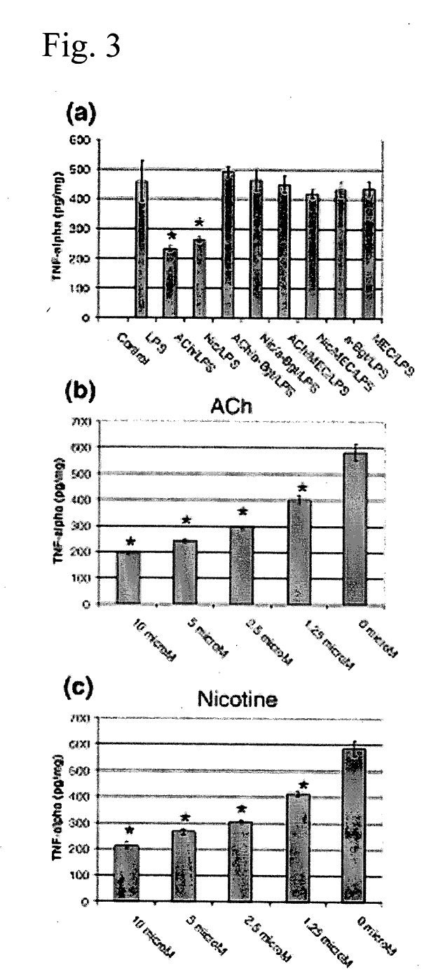

ACh or nicotine pre-treatment resulted in a marked reduction of LPS-induced TNF-α release (FIG. 3a). Co-pre-treatment with selective α7 nAChR antagonist, α-bungarotoxin or non-selective nAChR antagonist, mecamylamine, significantly blocked Ach- or nicotine-mediated inhibition of TNF-α production (FIG. 3a). Furthermore, ACh or nicotine pre-treatment alone inhibited TNF-α production in a dose-dependent manner (FIGS. 3b and c).

### EXAMPLE 2

To determine whether ACh or nicotine could inhibit LPS-induced TNF-α production in these microglial cells, the inventors pre-incubated microglial cells with ACh or nicotine for 30 minutes and challenged these cells with LPS for 4 hours. Data shows that the pretreatment with either ACh or nicotine results in a marked reduction of LPS-induced TNF-α release (FIG. 3A). As shown in FIG. 3A, primary cultured microglial cells were pre-stimulated for 30 minutes with ACh (5 μM, or nicotine (5 μM in the presence or absence of nACh antagonists [α-bungarotoxin (α-Bgt), 10 nM; mecamylamine (MEC), 10 μM) and challenged with LPS (100 ng/mL) for 4 hours. TNF-α release in cell-free supernatants was assayed by ELISA kit. ANOVA revealed significant main effects of an interaction between LPS and ACh plus LPS (ACh/LPS) (p<0.01) or nicotine plus LPS (Nic/LPS) (p<0.01). One-way ANOVA revealed significant between-groups differences (p<0.01), and post hoc testing showed significant differences between ACh/LPS and ACh/α-Bgt/LPS (p<0.01), and between Nic/LPS and Nic/α-Bgt/LPS (p<0.01), as well as between ACh/LPS and ACh/MEC/LPS (p<0.01), and between Nic/LPS and Nic/MEC/LPS (p<0.01). However, no significances were noted between LPS and α-Bgt/LPS (p>0.05), or between LPS and MEC/LPS (p>0.05). In addition, co-incubation with either α-bungarotoxin or nonselective nAChR antagonist, mecamylamine, significantly recovers ACh or nicotine-mediated inhibition of this microglial TNF-α production (FIG. 3A). Furthermore, this TNF-α production is also inhibited by ACh or nicotine pretreatment in a dose-dependent manner (from 1.25 μM to 10 μM) (FIGS. 3B and C). Primary cultured microglial cells pretreated with ACh (FIG. 3B) or nicotine (2C) at a range of dose as indicated for 30 minutes and challenged with LPS (100 ng/mL) for 4 hours. Microglial TNF-α release was measured by ELISA. ACh or nicotine pre-treatment dose-dependently inhibits microglial LPS-induced TNF-α production. One-way ANOVA across doses revealed significant between-groups differences (p<0.01).

The findings demonstrate that physiologically relevant concentrations of ACh and nicotine have the ability to modulate microglial TNF-α release evoked by LPS through activation of α7 nAChRs. This is the first report of functional nAChR α7 subunit on microglia involved in a previously unknown cholinergic pathway which regulates microglial activation. Microglial modulation by nAChRs represents a novel physiological mechanism for the reported neuroprotective properties of nicotinic drugs in animal models of neurodegenerative disease. In the peripheral nervous system, a non-neuronal cholinergic system is strongly expressed within different components of the immune system and appears to be involved in the regulation of host defense mechanisms and inflammation. For example, it has been recently shown that efferent vagus nerve stimulation attenuates the systemic inflammatory response to LPS in blood born macrophages and this effect is mediated by the principle vagal neurotransmitter ACh acting at α7 nAChR receptors. While the findings provide evidence for a similar role for ACh in regulation of inflammation in the brain, the source of ACh for microglial regulation remains unclear.

Macrophages and microglia exhibit differential proinflammatory responses to external stimuli. Watters, et al., recently reported on important fundamental differences between macrophages and microglia in their response to bacterial endotoxin, lypopolysaccharide (LPS)(see Watters, et al., 2002. LPS potently induces nitric oxide and interleukin-Ib production but fails to activate ERK-1 and ERK-2 in murine microglial cells. J. Biol. Chem. 277: 9077-9087). LPS is a component of the outer membrane of Gram-negative bacteria and promotes the activation of macrophages and microglia. Although these cells are highly LPS-responsive, they serve unique tissue-specific functions and exhibit different LPS sensitivities. The Watters study evaluated whether these biological differences reside in variations within LPS signaling pathways between these two cell types. Because the mitogen-activated protein kinases ERK-1 and ERK-2 have been implicated in the control of many immune responses, it was hypothesized that they are a key indicator for differences in macrophage and microglial LPS sensitivity. It was observed that murine RAW 264.7 macrophages and murine BV-2 microglial cells both respond to LPS by exhibiting increased IkappaBalpha degradation, enhanced NF-KappaB DNA binding activity, and elevated nitric oxide and interleukin-1β production. Although LPS potently stimulates ERk activation in RAW 264.7 macrophages, it does not activate ERK-1/-2 in BV-2 microglia. Moreover, antagonism of the MEK/ERK pathway potentiates LPS-stimulated nitric oxide production, suggesting that LPS-stimulated ERK activation can exert inhibitory effects in macrophage-like cells. These data support the idea that ERK activation is not a required function of LPS-mediated signaling events and illustrate that alternative, and or additional, pathways for LPS action exist between these two cell types.

It has been postulated that microglia can carry out both neurotrophic and neurotoxic functions in the brain and factors which determine which function microglia serve depend on a compilation of signals received from neighboring astrocytes and neurons. The present invention shows that signals related to the suppression of immunological properties of microglia by neurons in the healthy brain and the disruption from this physiological equilibrium in aging and diseases, may involve cholinergic communication via α7 receptors. Such an interpretation is consistent with the observation that cholinergic neurons decrease with age in parallel with increased microglial activation. Moreover, because astrocytes synthesize ACh as well as the potent α7 antagonist, kynurenic acid, astrocytes may also regulate microglial phagocytosis via a cholinergic signal. With this in mind, the present invention shows that the loss of cholinergic communication from damaged neurons and/or astrocytes may be partially responsible for the turning of microglia to a hyperactivated state, which allows them to escape neuronal control and to give rise to persistent inflammation, resulting in exacerbation of neurodegeneration.

The therapeutic compositions of the subject invention can be formulated according to known methods for preparing therapeutically useful compositions. A therapeutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question. The therapeutic composition can be adapted for various forms of administration. Administration can be continuous or at distinct intervals as can be determined by a person skilled in the art.

The administration of the cholinergic agonist is administered and dosed in accordance with good medical practice, taking into account the clinical condition of the individual patient, the site and method of administration, scheduling of administration, patient age, sex, body weight, and other factors known to medical practitioners.

The therapeutically “effective amount” for purposes herein is thus determined by such considerations as are known in the art.

A therapeutically effective amount of the cholinergic agonist is that amount necessary to provide a therapeutically effective amount of the compound in vivo. The amount of the cholinergic agonist, α-bungarotoxin, mecamylamine, or any combination thereof must be effective to achieve a response, including but not limited to total prevention of (e.g., protection against), improved survival rate or more rapid recovery, or improvement or elimination of symptoms associated with neurodegenerative disorders or other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

Administering or contacting, as used herein refers to the process of delivering to a cell, ex vivo, or a host, in vivo, a therapeutic substance, or a combination of several therapeutic substances. The process can include any method known in the art and is dependent on the type of substance or substances administered. Possible methods include, but are not limited to, parenteral (i.e. subcutaneously, intravenously, intramuscularly, intra-arterially, and direct injection into a tissue or organ), mucosal (i.e. intranasally), pulmonary (i.e. via inhalation), topical, via catheter (i.e. iontopheretically) or orally. Administration is usually achieved via a pharmaceutically acceptable carrier.

A cholinergic agonist, as used herein, is any compound that binds to cells expressing cholinergic receptor activity. One of skill in the art can readily determine whether any particular compound is a cholinergic agonist by any of several methods known in the art. Furthermore, any cholinergic agonist, now known or later discovered, would be expected to inhibit the release of proinflammatory cytokines from microglia. In a preferred embodiment, the cholinergic agonist is not otherwise toxic or harmful to the cell a therapeutically effective doses. In one embodiment, the anticholinergic is a naturally occurring compound produced by the patient or has been known in the art to be used therapeutically, including but no limited to acetylcholine, nicotine, galantamine, carbachol, levamisole, arecoline, muscarine, cevimeline, choline, cytisine, GTS-21, or derivatives thereof.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be the to fall therebetween. Now that the invention has been described,

What is claimed is:

1. A method of treating a neurodegenerative disorder in a patient, comprising the step of contacting a target cell with a therapeutically effective amount of a cholinergic antagonist wherein the target cell is a microglia.

2. The method of claim 1 wherein the cholinergic antagonist is chosen from the group consisting of acetycholine and nicotine.

3. The method of claim 1 wherein the cholinergic antagonist is administered concomitantly with an alpha-7 selective nicotinic antagonist.

4. The method of claim 3 wherein the nicotinic antagonist is chosen from the group consisiting of mecamylamine and alpha-bungarotoxin.

5. A method of treating a neurodegenerative disorder in a patient, comprising the step of contacting a microglia target cell with a therapeutically effective amount of a cholinergic antagonist selected from the group consisting of acetycholine and nicotine, the cholinergic antagonist administered concomitantly with an alpha-7 selective nicotinic antagonist.

6. The method of claim 5 wherein the nicotinic antagonist is chosen from the group consisting of mecamylamine and alpha-bungarotoxin.

7. A method if inhibiting the release of a proinflammatory cytokine from a cell, comprising the step of contacting the cell with a therapeutically effective amount of a cholinergic antagonist wherein the cell is a microglia.

8. The method of claim 7 wherein the cholinergic antagonist is chosen from the group consisting of acetycholine and nicotine.

9. The method of claim 7 wherein the cholinergic antagonist is administered concomitantly with an alpha-7 selective nicotinic antagonist.

10. The method of claim 9 wherein the nicotinic antagonist is chosen from the group consisting of mecamylamineand alpha-bungarotoxin.

11. The method of claim 7 wherein the proinfalammatory cytokine is Tumor Necrosis Factor (alpha).

12. A method if inhibiting the release of Tumor Necrosis Factor (alpha) from a cell, comprising the step of contacting the cell with a therapeutically effective amount of a cholinergic antagonist selected from the group consisting of acetycholine and nicotine, the cholinergic antagonist administered concomitantly with an α7 selective nicotinic antagonist wherein the cell is a microglia.

13. The method of claim 12 wherein the nicotinic antagonist is chosen from the group consisting of mecamylamine and alpha-bungarotoxin.
